(19) United States
(12) Patent Application Publication
(10) Pub. No.: US 2017/0014683 A1
(43) Pub. Date: Jan. 19, 2017

(54) DISPLAY DEVICE AND COMPUTER PROGRAM

(71) Applicant: Seiko Epson Corporation, Tokyo (JP)

(72) Inventors: Yuya MARUYAMA, Chino (JP); Hideki TANAKA, Chino (JP); Takayuki KITAZAWA, Suwa (JP)

(21) Appl. No.: 15/196,452

(22) Filed: Jun. 29, 2016

(30) Foreign Application Priority Data
Jul. 15, 2015 (JP) 2015-141083

(51) Int. Cl. A63B 24/00; G06F 3/01; A61B 5/11; G02B 27/01; A63B 23/16; G06F 3/00; G06T 19/00

(52) U.S. Cl. CPC A63B 24/0062; G06F 3/005; G06F 3/011; G06T 19/006; G02B 27/017; A63B 23/16; A61B 5/1125; G02B 2027/0138

(57) ABSTRACT

A display device includes a display section with which a pair of body portions performing cooperative exercise can be visually recognized, an imaging section that can image a marker attached to one body portion of the pair of body portions, and a display control section configured to cause the display section to display an image representing a normal motion of the other body portion of the pair of body portions. The display control section estimates, on the basis of the position of the captured marker, a position concerning the other body portion visually recognized in the display section in the cooperative exercise and causes the display section to display the image in the estimated position.

### BACKGROUND

1. Technical Field

The present invention relates to a display device and a computer program.

2. Related Art

As a rehabilitation device, there has been known a device that shows a paralyzed body portion to a patient (a user) as if the paralyzed body portion is moving. For example, in a rehabilitation device described in JP-A-2015-39522 (Patent Literature 1), a marker is stuck to a paralyzed hand and, by using a head-mounted display device, a moving image serving as a model of a motion is displayed in a display position of the hand recognized by the marker.

JP-A-2015-103010 (Patent Literature 2) is also an example of related art.

In the rehabilitation device described in Patent Literature 1, the marker needs to be stuck to the paralyzed hand of the patient. However, since the paralyzed hand is a disabled portion, it is not easy to attach the marker. It is likely that the marker prevents the movement of the hand and the patient cannot smoothly perform rehabilitation exercise. Besides, there have been demands for a reduction in the size, manufacturing, improvement of convenience of use, and the like of the device.

### SUMMARY

An advantage of some aspects of the invention is to solve at least a part of the problems described above, and the invention can be implemented as the following aspects.

(1) An aspect of the invention is directed to a display device including: a display section with which a pair of body portions performing cooperative exercise can be visually recognized; an imaging section that can image a marker attached to one body portion of the pair of body portions; and a display control section configured to cause the display section to display an image representing a normal motion of the other body portion of the pair of body portions. The display control section estimates, on the basis of the position of the captured marker, a position concerning the other body portion visually recognized in the display section in the cooperative exercise and causes the display section to display the image in the estimated position. With the display device according to this aspect, the display position of the image representing the normal motion of the other body portion having disability is determined from the position of the marker attached to the one body portion that is normal. Therefore, it is unnecessary to attach the marker to the disabled other body portion. Therefore, it is possible to solve a problem of the attachment of the marker. Further, it is possible to prevent a situation in which rehabilitation exercise is not smoothly performed because of the marker.

(2) In the display device according to the aspect, the display control section may store, in advance, reference information that can specify a relative position of the other body portion to the one body portion in the cooperative exercise and perform the estimation of the visually recognized position on the basis of the position of the captured marker and the reference information. With this configuration, it is possible to highly accurately estimate the position concerning the disabled body portion visually recognized in the display section in the cooperative exercise. Therefore, it is possible to further improve an illusion effect in which a user misapprehends that the image of the hand of the user.

(3) In the display device according to the aspect, the pair of body portions may be both hands, the cooperative exercise may be exercise for gripping an object to be gripped with both the hands, and the reference information may be the size of the object to be gripped. With the display device according to this aspect, it is possible more accurately superimpose the image on the disabled body portion.

(4) In the display device according to the aspect, the display section may be a head-mounted display section. With the display device according to this aspect, it is possible to further improve augmented reality by mounting the display device on a head.

(5) Another aspect of the invention is directed to a computer program. The computer program is a computer program for controlling a display device including a display section with which a pair of body portions performing cooperative exercise can be visually recognized and an imaging section that can image a marker attached to a normal body portion of the pair of body portions. The computer program causes a computer to realize a function of causing the display section to display an image representing a normal motion of a disabled body portion of the pair of body portions. The function estimates, on the basis of the position of the marker captured by the imaging section, a position concerning the disabled body portion visually recognized in the display section in the cooperative exercise and causes the display section to display the image in the estimated position. With the computer program according to this aspect, like the display device in the aspect explained above, it is possible to solve a problem of the attachment of the marker. Further, it is possible to prevent a situation in which rehabilitation exercise is not smoothly performed because of the marker.

### BRIEF DESCRIPTION OF THE DRAWINGS

The invention will be described with reference to the accompanying drawings, wherein like numbers reference like elements.

FIG. 1 is an explanatory diagram showing the configuration of a head-mounted display device (an HMD) according to an embodiment of the invention.

FIG. 2 is an explanatory diagram showing the configuration of a display section for left eye in detail.

FIG. 3 is a block diagram functionally showing the configuration of the HMD.

FIGS. 4A and 4B are explanatory diagrams showing sticking positions of markers.

FIG. 5 is an explanatory diagram showing a state of preparatory work.

FIG. 6 is a flowchart for explaining a former half portion of rehabilitation processing executed by a control device.

FIG. 7 is a flowchart showing a latter half portion of the rehabilitation processing executed by the control device.

FIG. 8 is an explanatory diagram showing an example of a message displayed in step S170.

FIG. 9 is an explanatory diagram showing a display screen visually recognized by a user in a state in which the user grabs a business card with a normal hand.

FIG. 10 is an explanatory diagram showing an example of an exercise model.

FIG. 11 is an explanatory diagram showing an example of an image visually recognized by the user during reproduction.

### DESCRIPTION OF EXEMPLARY EMBODIMENTS

An embodiment of the invention is explained below.

### A. Basic Configuration of an HMD

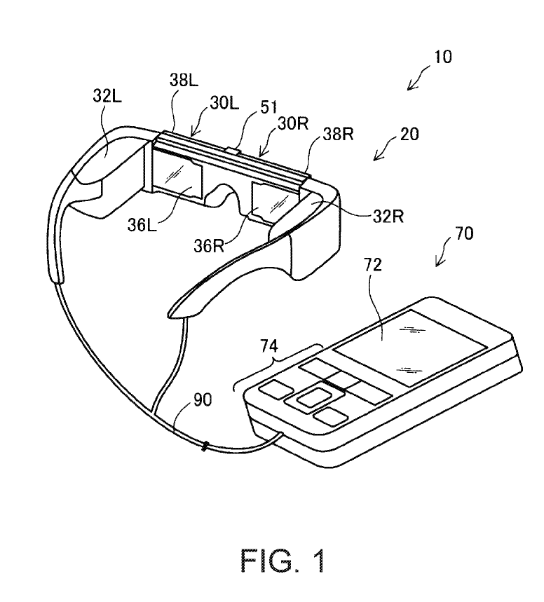

FIG. 1 is an explanatory diagram showing the configuration of a head-mounted display device 10 according to an embodiment of the invention. The head-mounted display device 10 is a display device mounted on a head and is also called head mounted display (HMD). The HMD 10 is a device for performing rehabilitation with one hand. In this embodiment, the HMD 10 is an optical transmission type (a see-through type) with which a user can visually recognize a virtual image and, at the same time, visually recognize a real space.

The HMD 10 includes a display device 20 having a shape like eyeglasses and a control device (a controller) 70. The display device 20 and the control device 70 are communicably connected by wire or radio. In this embodiment, the display device 20 and the control device 70 are connected by a wired cable 90. The control device 70 communicates a signal of an image (an image signal) and a signal of control (a control signal) to and from the display device 20 via the cable 90.

The display device 20 includes a display section for the left eye (a display section for left eye) 30L and a display section for the right eye (a display section for right eye) 30R.

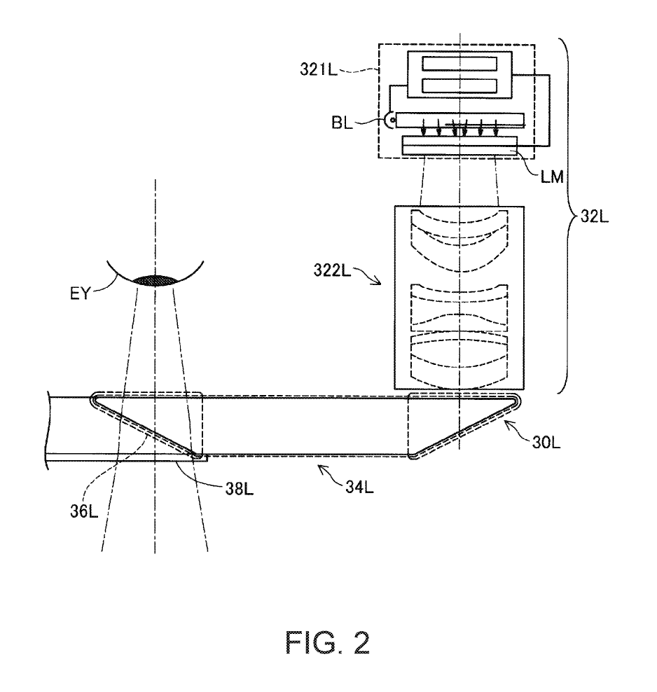

The display section for left eye 30L includes an image forming section for the left eye (an image forming section for left eye) 32L, a light guide section for the left eye (a light guide section for left eye 34L shown in FIG. 2), a reflecting section for the left eye (a reflecting section for left eye) 36L, and a shade for left eye 38L. The display section for right eye 30R includes an image forming section for the right eye (an image forming section for right eye) 32R, a light guide section for the light eye (same as the light guide section for left eye 34L shown in FIG. 2), a reflecting section for the right eye (a reflecting section for right eye) 36R, and a shade for right eye 38R.

FIG. 2 is an explanatory diagram showing the configuration of the display section for left eye 30L in detail. FIG. 2 is a view of the display section for left eye 30L viewed from right above. The image forming section for left eye 32L included in the display section for left eye 30L is disposed in a base portion of a temple of eyeglasses. The image forming section for left eye 32L includes an image generating section for the left eye (an image generating section for left eye) 321L and a projection optical system for the left eye (a projection optical system for left eye) 322L.

The image generating section for left eye 321L includes a light source of a backlight for the left eye (a backlight light source for left eye) BL and a light modulating element for the left eye (a light modulating element for left eye) LM. In this embodiment, the backlight light source for left eye BL includes a set of light sources for respective light emission colors such as red, green, and blue. As the light sources, for example, light emitting diodes (LEDs) and the like can be used. In this embodiment, the light modulating element LM includes a liquid crystal display device, which is a display element.

The display section for left eye 30L acts as explained below. When an image signal for the left eye is input to the image generating section for left eye 321L from the control device 70 (FIG. 1), the light sources of the backlight light source for left eye BL emit red light, green light, and blue light. The red light, the green light, and the blue light emitted from the light sources diffuse to be projected on the light modulating element for left eye LM. The light modulating element for left eye LM spatially modulates the projected red light, green light, and blue light according to the image signal input to the image generating section for left eye 321L from the control device 70 to thereby emit image light corresponding to the image signal.

The projection optical system for left eye 322L includes, for example, a projection lens group. The projection optical system for left eye 322L projects image light emitted from the light modulating element for left eye LM of the image generating section for left eye 321L and changes the image light to light beams of a parallel state. The image light changed to the light beams of the parallel state by the projection optical system for left eye 322L is projected on the light guide section for left eye 34L.

The light guide section for left eye 34L guides the image light from the projection optical system for left eye 322L to a predetermined surface (a semi-transmission reflection surface) of a triangular prism included in the reflecting section for left eye 36L. The front or the back of the semi-transmission reflection surface, which is formed in the reflecting section for left eye 36L, facing a left eye EY of the user during wearing is applied with reflection coating such as a mirror layer. The image light guided to the semi-transmission reflection surface formed in the reflecting section for left eye 36L is totally reflected toward the left eye EY of the user by the surface applied with the reflection coating. Consequently, image light corresponding to the guided image light is output from an area (an image extraction area) in a predetermined position of the reflecting section for left eye 36L. The output image light enters the left eye EY of the user and forms an image (a virtual image) on the retina of the left eye EY.

At least a part of light made incident on the reflecting section for left eye 36L from the real space is transmitted through the semi-transmission reflection surface formed in the reflecting section for left eye 36L and guided to the left eye EY of the user. Consequently, for the user, an image formed by the image forming section for left eye 32L and an optical image from the real space are seen as being superimposed.

The shade for left eye 38L is disposed on the opposite side of the left eye EY of the user in the light guide section for left eye 34L. In this embodiment, the shade for left eye 38L is detachable. The shade for left eye 38L is attached in a bright place or attached when the user desires to concentrate on a screen. Therefore, the user can clearly view the image formed by the image forming section for left eye 32L.

As shown in FIG. 1, the display section for right eye 30R includes a similar configuration symmetrical to the configuration of the display section for left eye 30L and acts in the same manner as the display section for left eye 30L. As a result, when the user wears the display device 20 on the head, for the user, an image corresponding to image light output from an image extraction area of the display device 20 (an image extraction area of the reflecting section for left eye 36L and an image extraction area of the reflecting section for right eye 36R) is seen as being displayed. Therefore, the user can recognize the image. At least a part of light from the real space is transmitted through the image extraction area of the display device 20 (the image extraction area of the reflecting section for left eye 36L and the image extraction area of the reflecting section for right eye 36R). Therefore, the user can view the real space while wearing the display device 20 on the head.

In this way, the user can simultaneously view (visually recognize) the image displayed on the image extraction area of the display device 20 (hereinafter simply referred to as “display image”) and the real space transmitted through the image extraction area. The display image serves as an AR image that gives augmented reality (AR) to the user.

In the display device 20, a camera 51 is provided in a position corresponding to the middle of the forehead of the user when the user wears the display device 20. Therefore, in a state in which the user wears the display device 20 on the head, the camera 51 picks up an image of the real space in a direction in which the user faces. The camera 51 is a monocular camera but may be a stereo camera.

The control device 70 is a device for controlling the display device 20. The control device 70 includes a touch pad 72 and an operation button section 74. The touch pad 72 detects contact operation on an operation surface of the touch pad 72 and outputs a signal corresponding to detection content. As the touch pad 72, various touch pads such as an electrostatic type, a pressure detection type, and an optical type can be adopted. The operation button section 74 includes various operation buttons, detects operation of the operation buttons, and outputs a signal corresponding to detection content. The touch pad 72 and the operation button section 74 are operated by the user.

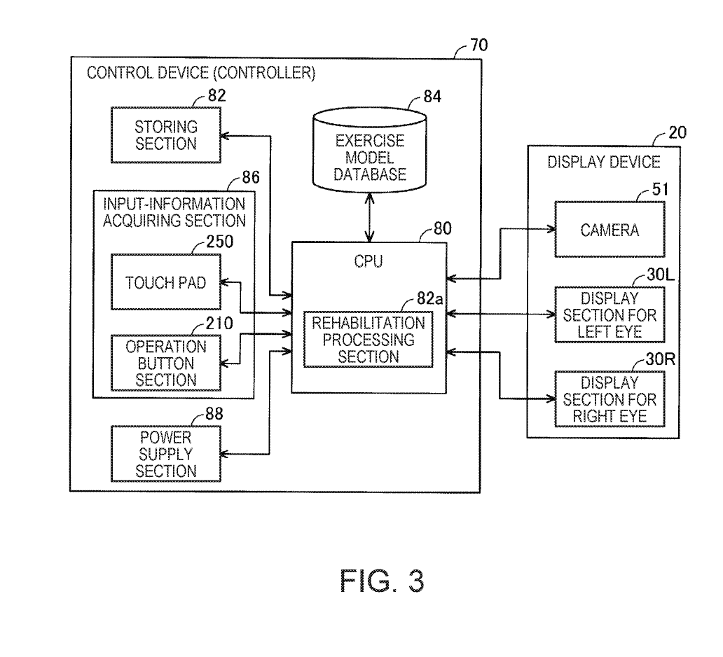

FIG. 3 is a block diagram functionally showing the configuration of the HMD 10. The control device 70 includes a CPU 80, a storing section 82, an exercise model database 84, an input-information acquiring section 86, and a power supply section 88. The sections are connected to one another by a bus or the like.

The storing section 82 includes a ROM, a RAM, a DRAM, or a hard disk. In the storing section 82, various computer programs such as an operating system (OS) are stored. In this embodiment, there is a computer program for rehabilitation as one of the stored computer programs.

The exercise model database 84 is a database in which exercise models are accumulated. The exercise model is moving image data obtained by modeling exercise set as a target in rehabilitation. In this embodiment, an exercise model for the left hand and an exercise model for the right hand are accumulated in advance. Note that the exercise model may be a collection of several still image data instead of the moving image data. Further, the exercise model may be data including a set of feature point positions of a hand. The exercise model can be replaced with any data as long as a moving image can be constructed from the data. Further, the exercise model may include parameters such as the number of times, speed, and the like of exercise.

The input-information acquiring section 86 includes the touch pad 72 and the operation button section 74. The input-information acquiring section 86 receives an input of a signal corresponding to the detection content received from the touch pad 72 or the operation button section 74.

The power supply section 88 supplies electric power to the components requiring the electric power in the control device 70 and the display device 20.

The CPU 80 reads out and executes the computer programs stored in the storing section 82 to thereby achieve various functions. Specifically, the CPU 80 achieves a function of executing, when detection content of operation is input from the input-information acquiring section 86, processing corresponding to the detection result, a function of reading data from and writing data in the storing section 82, and a function of controlling supply of electric power from the power supply section 88 to the components.

The CPU 80 reads out and executes the computer program for rehabilitation stored in the storing section 82 to thereby also function as a rehabilitation processing section 82a that executes rehabilitation processing. The rehabilitation processing is processing for displaying an AR image representing a normal motion of a disabled body portion (one hand) to thereby cause the user of the HMD 10 to perform cooperative exercise training. The CPU 80 and the rehabilitation processing section 82a, which is a function executed by the CPU 80, are equivalent to a subordinate concept of the “display control section”.

### B. Preparatory Work

In this embodiment, as a target person of rehabilitation, that is, the user of the HMD 10, a patient having disabled one hand and the other normal hand is assumed. As a disability, there is, for example, a paralysis due to a stroke. In the following explanation, a hand having disability is referred to as “disabled hand” and a hand without disability is referred to as “normal hand”. Note that “normal” does not need to be limited to a state without any disability and may be a state in which a hand functionally has slight disability.

In performing the cooperative exercise training using the HMD 10, the user needs to perform two kinds of preparatory work. First preparatory work is work for attaching markers. The markers are labels for designating a position where an AR image is displayed in the HMD 10.

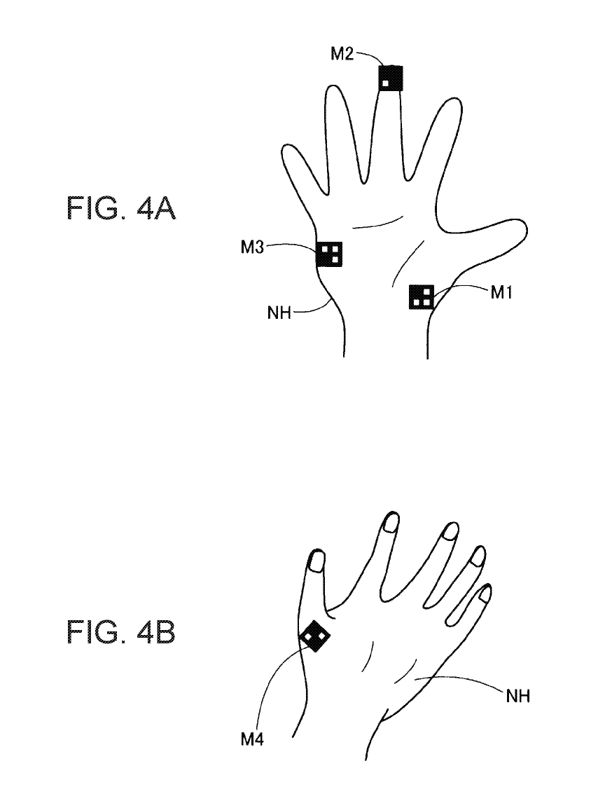

FIGS. 4A and 4B are explanatory diagrams showing sticking positions of markers. FIG. 4A shows the side of the palm of the normal hand. FIG. 4B shows the side of the back of the normal hand. It is assumed that the right hand is the normal hand. Four markers are prepared. As shown in FIG. 4A, first to third three markers M1, M2, and M3 are stuck to the side of the palm of a normal hand NH. Specifically, the first marker M1 is stuck to the base of the thumb (so-called mount of Venus) of the palm, the second marker M2 is stuck to the tip of the middle finger of the palm, and the third marker M3 is stuck to the bulge (so called mount of Mars) closer to the wrist under the little finger of the palm.

Note that the sticking positions of the markers M1 to M3 are positions suitable for specifying the outer edge of the normal hand NH and do not need to be limited to the example explained above. For example, the sticking position of the first marker M1 can be changed to the tip position of the thumb of the palm and the sticking position of the third marker M3 can be changed to the tip position of the little finger of the palm. The number of markers is not limited to three. For example, the number of markers can be set to seven in total by adding markers at the tip position of the thumb, the tip position of the index finger, and the tip position of the ring finger to the first to third markers M1 to M3 and can be set to two by sticking markers to the tip position of the thumb and the tip position of the little finger of the palm.

As shown in FIG. 4B, a fourth marker M4 is stuck between the thumb and the index finger on the side of the back of the normal hand NH. The sticking position of the fourth marker M4 is not limited to this and can be any position as long as the normal hand can be recognized in an initial posture of cooperative exercise training explained below. The marker on the side of the back of the normal hand NH is not limited to one marker and can be a plurality of markers.

The sticking of the markers M1 to M4 is performed by an assistant of rehabilitation. Note that, if the user can stick the markers M1 to M4 with the disabled left hand, the user may stick the markers by himself or herself.

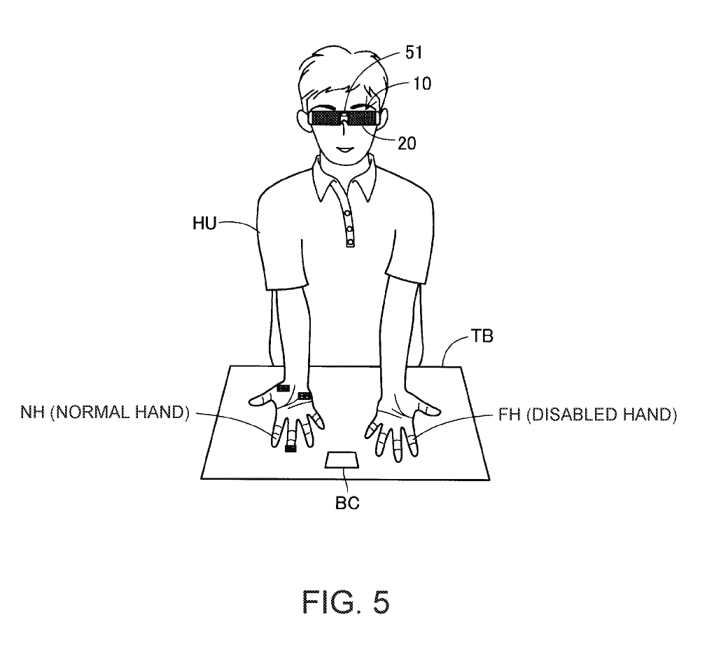

FIG. 5 is an explanatory diagram showing a state of second preparatory work. After finishing the first preparatory work, as the second preparatory work, a user HU is located in front of a rehabilitation table TB such as a desk or a table while wearing the display device 20 of the HMD 10 on the head. The user HU stretches out the left hand, which is a disabled hand FH, and the right hand, which is the normal hand NH, over the rehabilitation table TB. The normal hand NH is opened with the palm directed upward. “The hand is opened” is a state in which the joints of the fingers are stretched and the fingers are opened, that is, a so-called “paper” state in rock-paper-scissors game. The markers M1 to M4 are stuck to the normal hand NH by the first preparatory work. The disabled hand FH is in a natural state with the palm directed upward, that is, a state in which the joints of the fingers are slightly bent. In this embodiment, an object to be gripped, for example, a business card BC is placed on the rehabilitation table TB as a gadget for rehabilitation.

In the state shown in FIG. 5, the touch pad 72 and the operation button section 74 (FIG. 1) of the control device of the HMD 10 are operated, whereby execution of rehabilitation processing is instructed to the HMD 10. This operation is performed by, for example, the assistant of the rehabilitation. Note that the user may perform the operation using the normal hand and thereafter immediately set the normal hand in the state shown in FIG. 5.

### C. Rehabilitation Processing

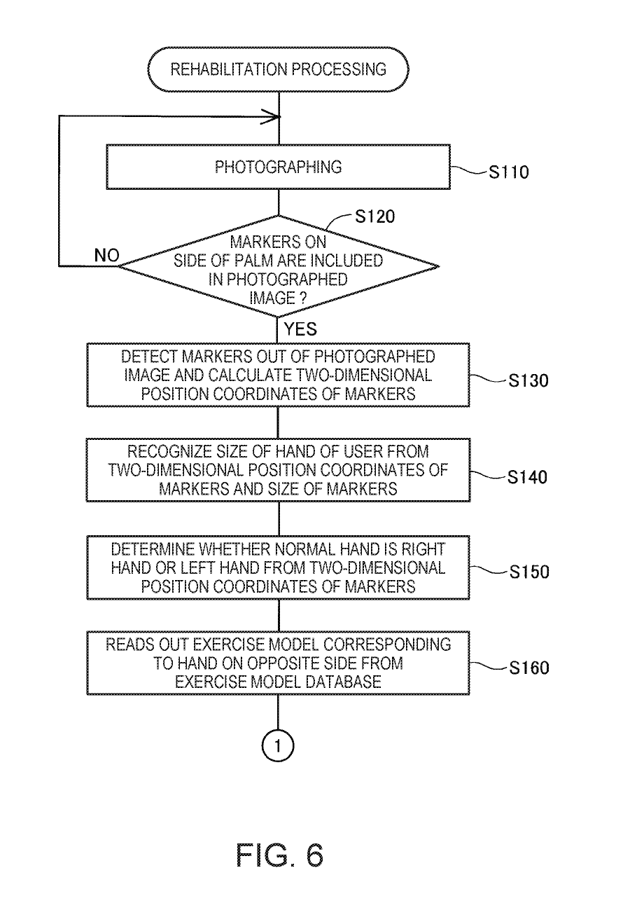

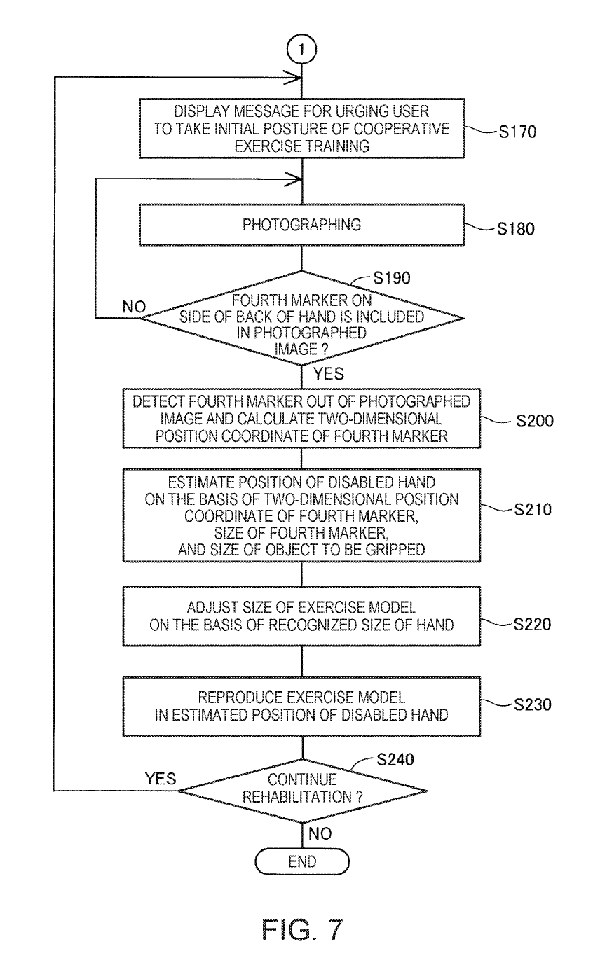

FIGS. 6 and 7 are flowcharts for explaining the rehabilitation processing executed by the control device 70. The rehabilitation processing is processing by the rehabilitation processing section 82a (FIG. 3). The execution of the rehabilitation processing is started by the CPU 80 when an instruction for the execution of the rehabilitation processing is received by the input-information acquiring section 86 (FIG. 3).

As shown in FIG. 6, when the processing is started, first, the CPU 80 performs imaging with the camera 51 (step S110). The CPU 80 determines whether the markers M1 to M3 stuck to the side of the palm are included in a captured image obtained by the imaging (step S120). “The markers M1 to M3 are included” means that all of the three markers M1 to M3 are included. When at least one of the markers M1 to M3 is not included, it is determined that the markers M1 to M3 are not included.

In the state shown in FIG. 5, in performing the rehabilitation, the user moves the eyes to the rehabilitation table TB on which the hands are placed. The camera 51 picks up an image of a real space in a direction in which the user faces. Therefore, when the user moves the eyes to the rehabilitation table TB, the markers M1 to M3 are included in the captured image by the camera 51. The determination in step S120 is affirmative. In the case of the affirmative determination, the CPU 80 advances the processing to step S130. On the other hand, if determining in step S120 that the markers M1 to M3 are not included, the CPU 80 returns the processing to step S110 and repeatedly executes the processing in steps S110 and S120.

In step S130, the CPU 80 detects the markers M1 to M3 out of the captured image obtained in step S110 and calculates two-dimensional position coordinates of the markers M1 to M3. A coordinate system indicating the two-dimensional position coordinates corresponds to a display screen by the display device 20. The three markers M1 to M3 specify the outer edge of the normal hand NH. Therefore, the spread of the two-dimensional position coordinates of the markers M1 to M3 is decided by the (actual) size of the hand of the user and the distance from the markers to the camera 51. The distance from the marker to the camera 51 can be calculated on the basis of the size in the captured image of any one marker among the three markers M1 to M3. Therefore, in the following step S140, the CPU 80 recognizes the (actual) size of the hand of the user on the basis of the two-dimensional position coordinates of the markers M1 to M3 calculated in step S130 and the size in the captured image of the one marker.

Subsequently, the CPU 80 determines from the two-dimensional position coordinates of the markers M1 to M3 calculated in step S130 whether the normal hand NH, to which the markers M1 to M3 are stuck, is the right hand or the left hand (step S150). The markers M1 to M4 can be individually identified. Therefore, it is possible to determine whether the normal hand NH is the right hand or the left hand according to whether the first marker M1 provided in the base of the thumb of the palm is located on the right side or the left side with respect to the third marker M3 provided closer to the wrist under the little finger of the palm. Note that this determination method is an example. Any method may be used as long as it is determined whether the normal hand NH is the right hand or the left hand according to a positional relation of the disposition of the markers M1 to M3.

Subsequently, the CPU 80 recognizes, as a disabled hand, the hand on the opposite side of the hand determined in step S150 and reads out an exercise model corresponding to the side of the disabled hand from the exercise model database 84 (step S160). That is, if determining in step S150 that the normal hand is the right hand, since the disabled hand is the left hand, the CPU 80 reads out an exercise model for the left hand. On the other hand, if determining in step S150 that the normal hand is the left hand, since the disabled hand is the right hand, the CPU 80 reads out an exercise model for the right hand. Details of the exercise model are explained below.

After executing step S160 in FIG. 6, the CPU 80 advances the processing to step S170 in FIG. 7. In step S170, the CPU 80 causes the display device 20 of the HMD 10 to display a message for urging the user to take an initial posture of the rehabilitation. The “initial posture” is a posture for gripping the business card BC with the normal hand NH.

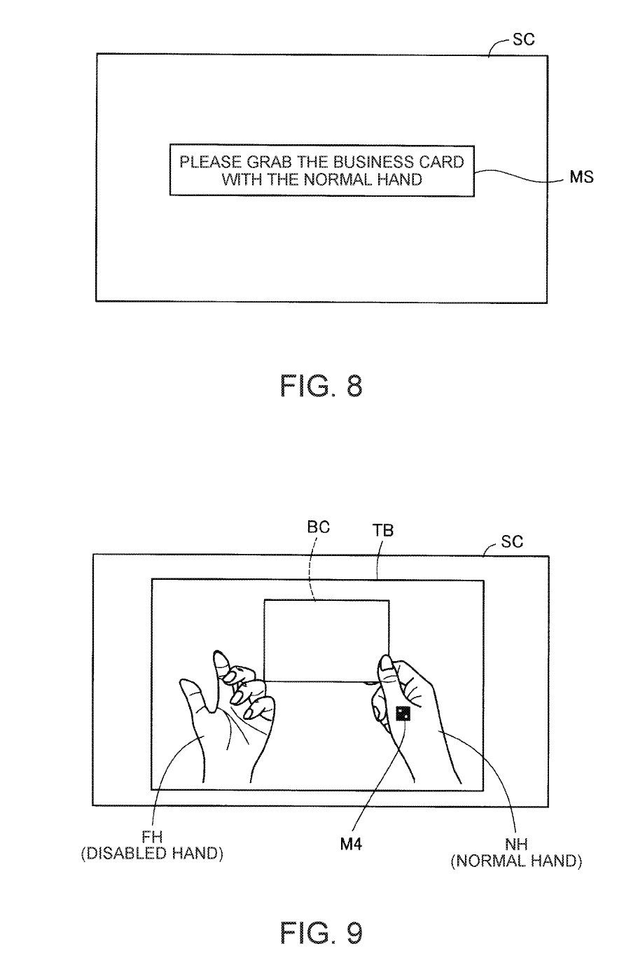

FIG. 8 is an explanatory diagram showing an example of the message displayed in step S170. SC in the figure indicates a display screen by the display device 20. In step S110, specifically, for example, a message MS “please grab the business card with the normal hand” is displayed on the display screen SC. The user visually recognizing the message MS on the display screen SC performs a motion of grabbing (gripping) the business card BC (FIG. 5) with the normal hand NH.

FIG. 9 is an explanatory diagram showing the display screen SC visually recognized by the user in a state in which the user grabs the business card BC with the normal hand NH. As shown in the figure, the user visually recognizes, in the display screen SC, as a real image of a real space transmitted through the display screen SC, the normal hand NH grabbing the business card BC and the disabled hand FH.

After executing step S170 (FIG. 7), the CPU 80 performs imaging with the camera 51 (step S180) and determines whether the fourth marker M4 stuck to the side of the back of the hand is included in a captured image obtained by the imaging (step S190). The fourth marker M4 is stuck between the thumb and the index finger on the side of the back of the normal hand NH. Therefore, when the user grabs the business card BC with the normal hand NH, the fourth marker M4 is included in the captured image by the camera 51. The determination in step S190 is affirmative. In the case of the affirmative determination, the CPU 80 advances the processing to step S200. On the other hand, if it is determined in step S190 that the fourth marker M4 is not included, the CPU 80 returns the processing to step S180 and repeatedly executes the processing in steps S180 and S190.

In step S200, the CPU 80 detects the fourth marker M4 out of the captured image obtained in step 5180 and calculates a two-dimensional position coordinate of the fourth marker M4. A coordinate system indicating the two-dimensional position coordinate corresponds to the display screen by the display device 20.

Subsequently, the CPU 80 estimates the position of the disabled hand on the basis of the two-dimensional position coordinate of the fourth marker M4 calculated in step S200, the size in the captured image of the fourth marker M4, and the (actual) size of the business card, which is the object to be gripped (step S210). In this embodiment, “the position of the disabled hand” is a position that the disabled hand (e.g., the left hand) can take when cooperative exercise for gripping the business card BC using the right hand and the left hand is performed. The two-dimensional position coordinate of the fourth marker M4 decides the position of the normal hand NH (e.g., the right hand). Therefore, it is determined that the disabled hand is present in a position apart from the two-dimensional position coordinate of the fourth marker M4 by the size in the captured image of the business card. The size in the captured image of the business card can be calculated on the basis of the (actual) size of the business card and the distance from the marker to the camera 51. Therefore, the position of the disabled hand visually recognized in the cooperative exercise is unconditionally decided with respect to the two-dimensional position coordinate of the fourth marker M4, the size in the captured image of the fourth marker M4, and the (actual) size of the business card.

In this embodiment, the two-dimensional position coordinate of the fourth marker M4 is represented as a variable X, the size in the captured image of the fourth marker M4 is represented as a variable Y, the (actual) size of the business card is represented as a constant C, the position of the disabled hand visually recognized in the cooperative exercise is represented as a variable Z, and a formula representing the variable Z with respect to the variables X and Y and the constant C is experimentally calculated by a simulation and stored in the storing section 82. In step S210, the CPU 80 calculates, using the formula, the position of the disabled hand visually recognized in the display device 20 in the cooperative exercise. The (actual) size of the business card is equivalent to a subordinate concept of the “reference information”.

After executing step S210 (FIG. 7), the CPU 80 adjusts, on the basis of the size of the hand of the user recognized in step S140, the size of the exercise model read out in step S160 (step S220).

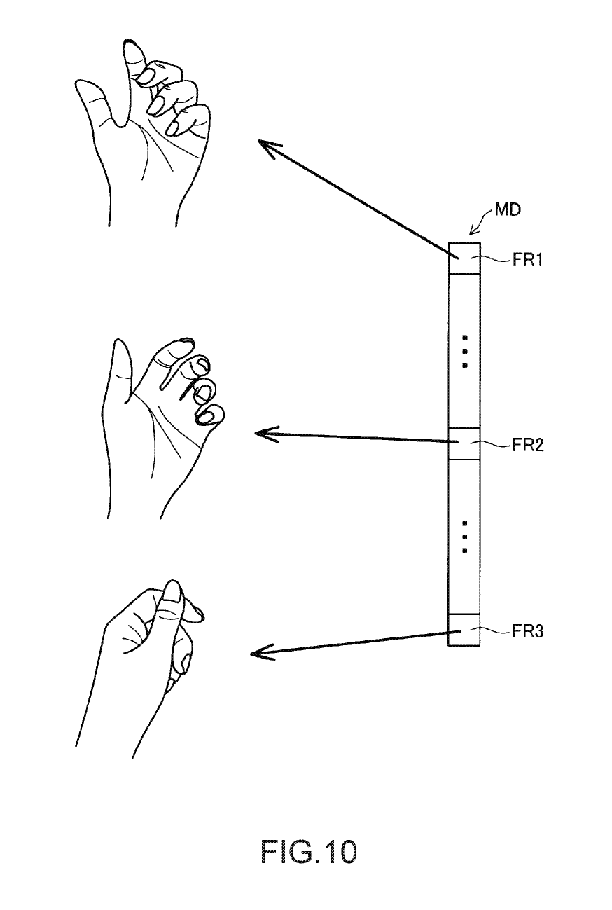

FIG. 10 is an explanatory diagram showing an example of the exercise model read out in step S160. An illustrated exercise model MD is an exercise model for the left hand. The exercise model MD is moving image data configured by a plurality of frames (still images) FR1, , FR2, , and FR3. One or a plurality of frames are included among the frames FR1 to FR3 as well.

The first frame FR1 represents a natural state in which the palm is directed upward. The state substantially coincides with the state of the disabled hand FH shown in FIG. 5. The last frame FR3 represents a state of the hand at the time when the hand grabs the business card BC (FIG. 5), which is the object to be gripped. The frame FR2 in the middle of the first frame FR1 and the last frame FR3 represents an intermediate state between the natural state in which the palm is directed upward and the state in which the hand grabs the business card.

With the exercise model MD configured as explained above, continuous exercise from the natural state in which the palm is directed upward to the state in which the hand grabs the business card, that is, exercise in gripping the business card is shown. In step S220, the CPU 80 adjusts the size of the exercise model MD on the basis of the size of the hand of the user recognized in step S140. That is, the exercise model stored in the exercise model database 84 (FIG. 3) has a general size of an adult. Therefore, in step S220, the CPU 80 performs size adjustment for enlarging or reducing the exercise model to match the size of the hand of the user.

Thereafter, the CPU 80 reproduces (displays) the exercise model adjusted in size in step S220 in the position of the disabled hand estimated in step S210 (step S230). The display is performed by causing the display section for left eye 30L and the display section for right eye 30R explained above to operate.

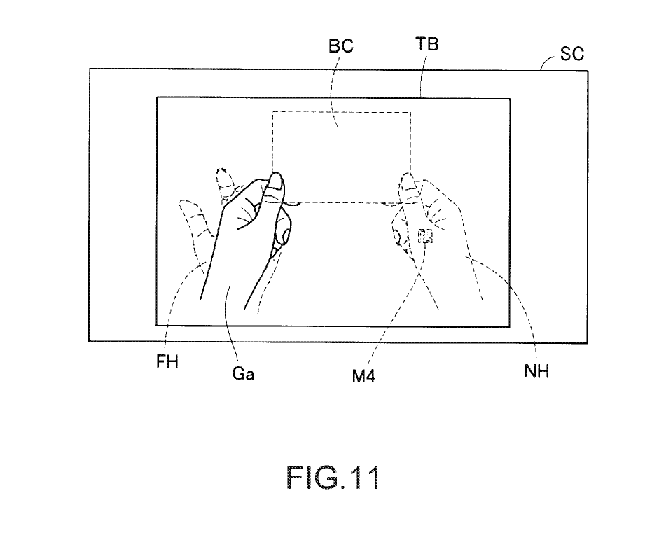

FIG. 11 is an explanatory diagram showing an example of an image visually recognized by the user during the reproduction. An image of the left hand indicated by a solid line in the figure is an image (an AR image) Ga of the reproduced exercise model. Images of the right hand and the left hand indicated by broken lines in the figure are the normal hand NH and the disabled hand FH of the user present in the real space seen through the display screen SC. As shown in the figure, for the user, the image Ga of the exercise model is superimposed on the disabled hand FH and visually recognized. In the example shown in the figure, the image Ga of the exercise model is a state in which the hand grabs the business card BC and is an image of the last frame FR3 shown in FIG. 10. From the state in which the hand is opened, the user opens and closes the hand following the movement of the image Ga of the exercise model to perform cooperative exercise training.

After executing step S230 in FIG. 7, the CPU 80 determines whether the rehabilitation is continued (step S240). The touch pad 72 and the operation button section 74 (FIG. 1) of the control device 70 are operated to instruct the HMD 10 to continue the rehabilitation. When receiving the instruction to continue the rehabilitation with the input-information acquiring section 86 (FIG. 2), the CPU 80 determines that the rehabilitation is continued. The operation of the touch pad 72 and the operation button section is performed by, for example, the assistant of the rehabilitation. Note that the user may perform the operation using the normal hand and thereafter immediately return the normal hand to the state shown in FIG. 5.

If determining in step S240 that the rehabilitation is continued, the CPU 80 returns the processing to step S170 and repeatedly executes the processing in steps S170 to S240. On the other hand, if determining in step S240 that the rehabilitation is not continued, the CPU 80 ends a routine of the rehabilitation processing.

### D. Effects of the Embodiment

With the HMD 10 in the embodiment configured as explained above, the display position of the AR image representing the normal motion of the disabled hand FH is determined from the positions of the markers M1 to M4 attached to the normal hand NH. Therefore, in this embodiment, it is unnecessary to attach markers to a disabled body portion. Therefore, it is possible to solve a problem of the attachment of the markers M1 to M4 and prevent a situation in which the rehabilitation exercise is not smoothly performed because of the markers M1 to M4.

As explained above (see FIG. 11), the user can superimpose the image Ga of the exercise model on the disabled hand FH and visually recognize the image Ga. Therefore, the user can perform the cooperative exercise training on an illusion that the image Ga of the exercise model is the hand of the user. Therefore, with the rehabilitation device 100 in this embodiment, it is possible to improve an effect of relieving a paralysis of a hand with the illusion effect.

### E. Modifications

The invention is not limited to the embodiment and the modifications thereof and can be carried out in various forms without departing from the spirit of the invention. For example, modifications explained below are also possible.

### Modification 1

In the embodiment, the cooperative exercise is the motion of gripping the object to be gripped using the right hand and the left hand. On the other hand, as a modification, the cooperative exercise may be a motion of beating a drum using the right hand and the left hand, a motion of joining the right hand and the left hand, or a motion of hitting a keyboard using the right hand and the left hand. The cooperative exercise does not need to be limited to the a motion of using the right hand and the left hand and may be a motion of using the right arm and the left arm, the right foot and the left foot (from the ankles to the toes), the right leg and the left leg (from the ankle to the pelvis), or the like. In the embodiment, the pair of body portions is the symmetrical body portions having the same functions. However, the pair of body portions is not limited to this and may be the right hand and the left arm, the right hand and the left foot, the right hand and the left leg, or the like. In the cooperative exercise of the body portions, if the position of one body portion of the pair of body portions is determined, it is possible to estimate the position of the other body portion. Therefore, it is possible to achieve action and effects same as those of the embodiment.

### Modification 2

In the embodiment, the object to be gripped used in the cooperative exercise is the business card. However, the object to be gripped may be an object of another shape such as a ruler or a tray instead of the business card. An instrument to be used does not need to be limited to the object to be gripped and can be replaced with objects held in various states such as an object held in a grabbed state. The cooperative exercise may be cooperative exercise performed without using an instrument. Note that, when the cooperative exercise is performed using an instrument, the size of the instrument is stored as the reference information.

### Modification 3

In the embodiment, the three markers are stuck to the side of the palm, the size of the hand of the user is recognized from the markers, and the size of the exercise model is adjusted on the basis of the size of the hand. On the other hand, as a modification, a configuration may be adopted in which the markers are not stuck to the side of the palm and the adjustment of the size of the exercise model is not performed. That is, the display position of the AR image may be determined using only the fourth marker M4 attached to the side of the back of the hand.

### Modification 4

In the embodiment, the HMD is a transmission-type display device in which the visual field of the user is not blocked in the mounted state of the HMD. On the other hand, as a modification, the HMD may be a non-transmission-type display device in which the visual field of the user is blocked. In the non-transmission-type HMD, an image of the real space is captured by a camera and an AR image is superimposed on the captured image. In the embodiment, the HMD includes the display section for left eye and the display section for right eye. However, the HMD may include only a display section for one eye instead of the display section for left eye and the display section for right eye.

### Modification 5

In the embodiment and the modifications, as the display device that can display the AR image, the head-mounted display device mounted on the head of the user is used. However, the display device is not limited to this. Various modifications of the display device are possible. For example, like a display device supported by an arm mounted on the shoulder or the neck of the user, a body-mounted display device mounted on the body of the user such as the head, the shoulder, or the neck may be used. The display device may be a display device of a placed type placed on a table or the like rather than being mounted on the user.

### Modification 6

In the embodiment and the modifications, the rehabilitation processing section 82a (FIG. 3) is explained as being realized by the CPU 80 executing the computer program stored in the storing section 82. However, the rehabilitation processing sect ion may be configured using an ASIC (Application Specific Integrated Circuit) designed to realize the function of the rehabilitation processing section.

### Modification 7

In the embodiment and the modifications, the camera 51 is integrally attached to the display device 20. However, the display device 20 and the camera 51 may be separately provided.

The invention is not limited to the embodiment, the examples, and the modifications and can be realized in various configurations without departing from the spirit of the invention. For example, the technical features in the embodiment, the examples, and the modifications corresponding to the technical features in the aspects described in the summary can be substituted or combined as appropriate in order to solve apart or all of the problems explained above or achieve a part of all of the effects explained above. Unless the technical features are explained as essential technical features in this specification, the technical features can be deleted as appropriate.

The entire disclosure of Japanese Patent Application No. 2015-141083 filed Jul. 15, 2015 is expressly incorporated by reference herein.

What is claimed is:

1. A display device comprising:
a display section with which a pair of body portions performing cooperative exercise can be visually recognized;
an imaging section that can image a marker attached to one body portion of the pair of body portions; and
a display control section configured to cause the display section to display an image representing a normal motion of the other body portion of the pair of body portions, wherein
the display control section estimates, on the basis of a position of the captured marker, a position concerning the other body portion visually recognized in the display section in the cooperative exercise and causes the display section to display the image in the estimated position.

2. The display device according to claim 1, wherein the display control section stores, in advance, reference information that can specify a relative position of the other body portion to the one body portion in the cooperative exercise and performs the estimation of the visually recognized position on the basis of the position of the captured marker and the reference information.

3. The display device according to claim 2, wherein
the pair of body portions is both hands,
the cooperative exercise is exercise for gripping an object to be gripped with both the hands, and
the reference information is a size of the object to be gripped.

4. The display device according to claim 1, wherein the display section is a head-mounted display section.

5. The display device according to claim 1, wherein
the one body portion is a normal body portion, and
the other body portion is a disabled body portion.

6. A computer-readable storage medium storing a program for controlling a display device including a display section with which a pair of body portions performing cooperative exercise can be visually recognized and an imaging section that can image a marker attached to the body portion of the pair of body portions,
the computer-readable storage medium storing a program causing a computer to realize a function of causing the display section to display an image representing a normal motion of the other body portion of the pair of body portions, wherein
the function estimates, on the basis of a position of the marker captured by the imaging section, a position concerning the other body portion visually recognized in the display section in the cooperative exercise and causes the display section to display the image in the estimated position.

7. The computer-readable storage medium storing a program according to claim 6, wherein
the one body portion is a normal body portion, and
the other body portion is a disabled body portion.
